(19) United States
(12) Patent Application Publication
(10) Pub. No.: US 2010/0028878 A1
(43) Pub. Date: Feb. 4, 2010

(54) Modulators of UDP-glucose ceramide glucosyltransferase for treating acne or hyperkeratinization

(75) Inventors: Fernand Labrie, Quebec (CA); Michel Rivier, Nice (FR); Van Luu-The, Charny (CA); Ezquiel L. Calvo, Quebec (CA); Irina Safonova, Nice (FR); Isabelle Carlavan, Grasse (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, Biot (FR)

(21) Appl. No.: 12/320,168

(22) Filed: Jan. 21, 2009

Related U.S. Application Data
(63) Continuation of application No. PCT/FR2007/051684, filed on Jul. 18, 2007.

(30) Foreign Application Priority Data
Jul. 19, 2006 (FR) 0653030

(51) Int. Cl. C12Q 1/68; G01N 33/53

(52) U.S. Cl. 435/6; 435/7.1

(57) ABSTRACT

An in vitro method for screening candidate compounds for the preventive or curative treatment of acne, includes the determination of the capacity of a compound to modulate the expression or the activity of UDP-glucose ceramide glucosyltransferase (UGCG), and the use of modulators of the expression or activity of this enzyme for the treatment of acne or skin disorders associated with a hyperkeratinization; methods for the in vitro diagnosis or prognosis of these pathologies are also described.

### CROSS-REFERENCE TO PRIORITY/PCT APPLICATIONS

This application claims priority under 35 U.S.C. §119 of FR 0653030, filed Jul. 19, 2006, and is a continuation/national phase of PCT/FR 2007/051684, filed Jul. 18, 2007, and designating the United States (published in the French language on Jan. 24, 2008 as WO 2008/009857 A2; the title and abstract were also published in English), each hereby expressly incorporated by reference in its entirety and each assigned to the assignee hereof.

### BACKGROUND OF THE INVENTION

1. Technical Field of the Invention

The present invention relates to the identification and administration of UDP-glucose ceramide glucosyltransferase modulating compounds for the treatment of acne and skin disorders associated with a hyperkeratinization. This invention also relates to methods for the in vitro diagnosis or prognosis of these pathologies.

2. Description of Background and/or Related and/or Prior Art

Acne is generally due to the involvement of three factors:

an excessive production of sebum (hyperseborrhea), under the influence of hormones and puberty,

a thickening of the skin (hyperkeratinization) whose pores and more particularly sebaceous glands become blocked, causing the formation of blackheads and comedones, and

the development of bacteria, causing inflammation and the appearance of red or white spots which are often painful.

The cornification of the keratinocytes is a complex process which involves the degradation of a large number of intracellular components. This process constitutes the final stage of epidermal differentiation and is associated with the formation of organized lamellar bilayers enriched in ceramides, cholesterol and fatty acids. The formation of ceramides is a key factor which leads to the formation of a normal stratum corneum and makes it possible to regulate the barrier function of the skin and desquamation (Holleran W M et al., J. Lipid Res., 1994, 35, 905-912). The reduction in the level of ceramides of the stratum corneum and the barrier function is observed in acne patients (Yamamoto A et al., Arch. Dermatol. Res., 1995, 187, 214-218). It has been shown that the topical application of retinoids or the oral administration of isotretinoin increases the level of ceramides in acne patients. The increase in ceramides is correlated with a decrease in comedones after treatment with retinoids applied topically (Melnic B et al., Arch. Dermatol. Res., 1988, 280, 97-102; Thielnitz A, Br. J. Dermatol., 2001, 1995, 95, 2903-2909). The retinoids are generally highly irritant and stripping compounds which cause redness in the region of the face that is not very aesthetic.

Need therefore exists to identify novel active compounds, the therapeutic profile of which will be similar, but with reduced side effects.

### SUMMARY OF THE INVENTION

It has now been discovered that the gene encoding UDP-glucose ceramide glucosyltransferase (UGCG) was expressed in the epidermis and in the human sebaceous glands, and that its expression was regulated by androgens, in vivo, in a mouse preputial gland model. Thus, targeting the UGCG gene or its expression product is now proposed to prevent and/or improve acne and/or any skin disorder associated with a hyperkeratinization.

The expression acne means all the forms of acne, namely, in particular acne vulgaris, comedo type acne, polymorphic acne, nodulocystic acne, acne conglobata, or secondary acnes such as solar acne, acne medicamentosa or occupational acne.

This invention also provides in vitro diagnostic or in vitro prognostic methods based on the detection of the expression or of the activity of UGCG.

### BRIEF DESCRIPTION OF THE DRAWINGS

FIGS. 1A and 1B are graphs showing the measurement of the expression of the UGCG gene in certain gonadectomized male mice, and

FIG. 2 represents the relative level of expression of the mRNA in certain male mice as a function of time.

### DETAILED DESCRIPTION OF BEST MODE AND SPECIFIC/PREFERRED EMBODIMENTS OF THE INVENTION

UGCG:

The enzyme UGCG denotes UDP-glucose ceramide glucosyltransferase. This enzyme is involved in the keratinization process. This process constitutes the final stage of epidermal differentiation, and is associated with the formation of a highly organized lamellar double layer enriched in ceramides, cholesterols and free fatty acids. These lipids are derived from the epidermal lamellar body, secretory organelles containing phospholipids, glucosylceramides and also hydrolytic enzymes. UDP-glucose ceramide glucosyltransferase is the enzyme responsible for the formation of ceramides from the cellular pool of glucosylceramides. It has been demonstrated that the production of ceramides is a critical step allowing the formation of normal stratum corneum and thereby regulates the permeable barrier and the desquamation of the skin (Hollerman W M et al., J Lipid Res., 1994, 35:905-912). A defect in UDP-glucose ceramide glucosyltransferase causes skin abnormalities described in patients suffering from Gaucher's disease. Recently, a novel therapeutic protocol was proposed for the management of Gaucher's disease. This approach is aimed at reducing the biosynthesis of glucosylceramide by administering inhibitors of glucosylceramide synthase. One of these inhibitors, N-butyldeoxynojirimycin (Miglustat), was recently approved by the FDA for the treatment of Gaucher's disease. The effect of the treatment with miglustat on acne has not been studied up until now.

In addition to their structural properties, glycosylceramides and ceramides appear as regulators of cell proliferation and differentiation. Studies in vitro have shown that changes in the level of glucosylceramides stimulated keratinocyte proliferation (Uchida Y et al., J Invest Dermatol., 1994, 102: 594a; Marsh N L et al, J Clin Invest. 1995, 95:2903-2909).

In the context of the present invention, the term “UGCG gene” or “UGCG nucleic acid” means the gene or nucleic acid sequence which encodes UDP-glucose ceramide glucosyltransferase. If the intended target is preferably the human gene or its expression product, this invention may also call into play cells expressing a heterologous UDP-glucose ceramide glucosyltransferase, through genomic integration or transient expression of an exogenous nucleic acid encoding the enzyme.

A human cDNA sequence for UGCG is reproduced in the annex (SEQ ID No. 1). It is the sequence NM003358.1 whose coding moiety is located from acid 291 to 1475.

Diagnostic Applications:

The present invention features an in vitro method for the diagnosis or monitoring of the progression of acne lesions or of a skin disorder associated with a hyperkeratinization in a subject, comprising comparing the expression or the activity of the protein UDP-glucose ceramide glucosyltransferase (UGCG), the expression of its gene or the activity of at least one of its promoters, in a biological sample from a subject compared with a biological sample from a control subject.

The expression of the UGCG protein may be determined by an assay of this protein by radioimmunoassay, for example by ELISA assay. Another method, in particular for measuring the expression of the UGCG gene, is to measure the quantity of corresponding mRNA, by any method as described above. An assay of the activity of the UGCG protein may also be employed.

In the context of a diagnosis, the “control” subject is a “healthy” subject.

In the context of a monitoring of the progression of acne lesions or of a skin disorder linked to a hyperkeratinization, the “control subject” refers to the same subject at a different time, which preferably corresponds to the start of the treatment (To). This measurement of the difference in the expression or the activity of the UGCG protein, of the expression of its gene or of the activity of at least one of its promoters, makes it possible in particular to monitor the efficacy of a treatment, in particular a treatment with a UGCG modulator, as indicated above or with another treatment against acne or a skin disorder associated with a hyperkeratinization. Such a monitoring can reassure the patient regarding the justification or the need for pursuing this treatment.

The present invention also features an in vitro method for determining the predisposition of a subject to develop acne lesions or a skin disorder associated with a hyperkeratinization, comprising comparing the expression or the activity of the UGCG protein, the expression of its gene or the activity of at least one of its promoters, in a biological sample from a subject compared with a biological sample from a control subject.

Here again, the expression of the UGCG protein may be determined by an assay of this protein by radioimmunoassay, for example by ELISA assay. Another method, in particular for measuring the expression of the UGCG gene, is to measure the quantity of corresponding mRNA by any method as described above. An assay of the activity of UGCG may also be employed.

The subject tested is here an asymptomatic subject with no skin disorder linked to a hyperkeratinization or an acne. The “control” subject in this method means a “healthy” reference subject or population. The detection of this predisposition allows the putting in place of a preventive treatment and/or an increased monitoring of the signs linked to acne or to a skin disorder associated with a hyperkeratinization.

In these in vitro diagnostic or prognostic methods, the biological test sample may be any biological fluid sample or a sample of a biopsy. Preferably, the sample may be a preparation of skin cells obtained for example by desquamation or biopsy. It may also be sebum.

Screening Methods:

This invention also features an in vitro method for screening candidate compounds for the preventive and/or curative treatment of acne, or of the skin disorders associated with a hyperkeratinization, comprising determining the capacity of a compound to modulate the expression or activity of UDP-glucose ceramide glucosyltransferase or the expression of its gene or the activity of at least one of its promoters, the said modulation indicating the usefulness of the compound for the preventive or curative treatment of acne or of the skin disorders associated with a hyperkeratinization. The method therefore makes it possible to select the compounds capable of modulating the expression or activity of the UDP-glucose ceramide glucosyltransferase, or the expression of its gene or the activity of at least one of its promoters.

More particularly, this invention features an in vitro method for screening candidate compounds for the preventive and/or curative treatment of acne or skin disorders associated with a hyperkeratinization, comprising the following steps:

a) preparing at least two biological samples or reaction mixtures;

b) bringing one of the samples or reaction mixtures into contact with one or more test compounds;

c) measuring the expression or activity of the protein UDP-glucose ceramide glucosyltransferase, the expression of its gene or the activity of at least one of its promoters, in biological samples or reaction mixtures;

d) selecting the compounds for which a modulation of the expression or activity of the UDP-glucose ceramide glucosyltransferase, or a modulation of the expression of its gene or a modulation of the activity of at least one of its promoters, is measured in the sample or mixture treated in b), compared with the untreated sample or mixture.

The expression “modulation” means any effect on the expression or activity of the enzyme, namely, optionally a partial or complete stimulation, but preferably a partial or complete inhibition. Thus, the compounds tested in step d) above preferably inhibit the expression or activity of the UGCG protein, the expression of its gene or the activity of at least one of its promoters. The difference in expression obtained with the test compound compared with a control prepared in the absence of the compound is significant from 25% or more.

In the present text, unless otherwise specified, “expression of a protein” means the quantity of this protein.

The expression “activity of a protein” means its biological activity.

The expression “activity of a promoter” means the capacity of this promoter to trigger the transcription of the DNA sequence coded downstream of this promoter (and therefore indirectly the synthesis of the corresponding protein).

The test compounds may be of any type. They may be of a natural origin or may have been produced by chemical synthesis. This may be a library of structurally defined chemical compounds, non-characterized compounds or substances or a mixture of compounds.

Various techniques may be used to test these compounds and identify the compounds of therapeutic interest, modulators of the expression or the activity of UDP-glucose ceramide glucosyltransferase.

According to a first embodiment, the biological samples are cells transfected with a reporter gene that is operably linked to all or part of the promoter of the UGCG gene, and step c) described above consists in measuring the level of expression of the said reporter gene.

The reporter gene may in particular encode an enzyme which, in the presence of a given substrate, leads to the formation of colored products, such as CAT (chloramphenicol acetyltransferase), GAL (beta-galactosidase) or GUS (beta-glucuronidase). This may also be the luciferase gene or GFP (Green Fluorescent Protein). The assay of the protein encoded by the reporter gene, or its activity, is carried out in a conventional manner by calorimetric, fluorometric or chemiluminescent techniques, among others.

According to a second embodiment, the biological samples are cells expressing the UGCG gene encoding UDP-glucose ceramide glucosyltransferase, and step c) above entails measuring the expression of the said gene.

The cell employed here may be of any type. This may be a cell endogenously expressing the UGCG gene, such as, for example, a liver cell, an ovarian cell or even better a keratinocyte or a sebocyte. It is also possible to employ organs of human or animal origin, such as for example the preputial gland, clitorial gland or sebaceous gland of the skin.

This may also be a cell transformed with a heterologous nucleic acid encoding a UDP-glucose ceramide glucosyltransferase, preferably of human origin, or of mammalian origin.

A wide variety of host cell systems may be employed, such as, for example, Cos-7, CHO, BHK, 3T3, HEK293 cells. The nucleic acid may be stably or transiently transfected by any method known to one skilled in this art, for example using calcium phosphate, DEAE-dextran, liposome, viruses, electroporation or microinjection.

In these methods, the expression of the UGCG gene or of the reporter gene may be determined by evaluating the level of transcription of the said gene, or its level of translation.

The expression level of transcription of a gene means the quantity of mRNA produced. The expression level of translation of a gene means the quantity of protein produced.

One skilled in this art is familiar with techniques allowing the quantitative or semi-quantitative detection of the mRNA of a gene of interest. The techniques based on the hybridization of mRNA with specific nucleotide probes are the most common (Northern Blot, RT-PCR, protection using RNase). It may be advantageous to employ detection markers such as fluorescent, radioactive or enzymatic agents or other ligands (for example avidin/biotin).

In particular, the expression of the gene may be measured by real-time PCR or by protection using RNase. The expression protection using RNase means the detection of a known mRNA among poly(A) RNAs of a tissue, which may be carried out with the aid of a specific hybridization with a labeled probe. The probe is a labeled (radioactive) complementary RNA for the messenger to be detected. It may be constructed from a known mRNA whose cDNA, after RT-PCR, has been cloned into a phage. The poly(A) RNA of the tissue where the sequence is to be detected is incubated with this probe under slow hybridization conditions in liquid medium. RNA:RNA hybrids are formed from the mRNA to be detected and the anti-sense probe. The hybridized medium is then incubated with a mixture of ribonucleases specific for single-stranded RNA, such that only the hybrids formed with the probe can withstand this digestion. The product of digestion is then deproteinized and repurified before being analyzed by electrophoresis. The labeled hybridized RNAs are detected by autoradiography.

The level of translation of the gene is evaluated for example by immunological assay of the product of the said gene. The antibodies employed for this effect may be of the polyclonal or monoclonal type. Their production involves conventional techniques. An anti-UDP-glucose ceramide glucosyltransferase polyclonal antibody may, inter alia, be obtained by immunization of an animal such as a rabbit or a mouse, with the whole enzyme. The antiserum is collected and then depleted according to methods known per se by one skilled in this art. A monoclonal antibody may, inter alia, be obtained by the conventional Kôhler and Milstein method (Nature (London), 256: 495-497 (1975)). Other methods of preparation of monoclonal antibodies are also known. It is possible, for example, to produce monoclonal antibodies by expressing a nucleic acid cloned from a hybridoma. It is also possible to produce antibodies by the phage display technique by introducing antibody cDNAs into vectors, which are typically filamentous phages which display V gene libraries at the surface of the phage (for example fUSE5 for E. coli).

The immunological assay may be carried out in a solid phase or in a homogeneous phase; in a single stage or in two stages; as a sandwich method or as a competitive method, by way of non-limiting examples. According to a preferred embodiment, the capture antibody is immobilized on a solid phase. It is possible to employ, by way of non-limiting examples of a solid phase, microplates, in particular polystyrene microplates, or solid particles or beads, paramagnetic beads.

ELISA assays, radioimmunoassays or any other detection technique may be carried out in order to reveal the presence of the antigen-antibody complexes formed.

The characterization of the antigen-antibody complexes, and more generally of the isolated or purified proteins, but also recombinant proteins (obtained in vivo and in vitro), may be carried out by mass spectrometry analysis. This identification is made possible by virtue of the analysis (determination of the mass) of peptides generated by the enzymatic hydrolysis of the proteins (trypsin in general). Generally, the proteins are isolated according to methods known to one skilled in this art, prior to the enzymatic digestion. The analysis of the peptides (in hydrolysate form) is performed by separation of the peptides by HPLC (nano-HPLC) based on their physicochemical properties (reversed phase). The determination of the mass of the peptides thus separated is carried out by ionization of the peptides or by direct coupling to mass spectrometry (electrospray ESI mode), or after deposition and crystallization in the presence of a matrix known to one skilled in this art (analysis in MALDI mode). The proteins are then identified using appropriate software (for example, Mascot).

According to a third embodiment, step a) described above entails preparing reaction mixtures each comprising an enzyme UDP-glucose ceramide glucosyltransferase and a substrate of the enzyme, and step c) described above entails measuring the enzyme activity.

The enzyme may be produced according to customary techniques using Cos-7, CHO, BHK, 3T3 and HEK293 cells. It may also be produced with the aid of microorganisms such as bacteria (for example, E. coli or B. subtilis), yeasts (for example Saccharomyces, Pichia) or insect cells, such as Sf9 or Sf21.

The determination of the enzymatic activity preferably comprises the determination of the transferase activity, by extraction of the lipids produced and chromatographic analysis.

Assays of the enzymatic activity of UGCG are described in the literature (see for example Futerman et al., 1991, Biochem J., 280, 295-302).

Thus, the activity of UDP-glucose ceramide glucosyltransferase may be evaluated in the following manner: liver fractions are incubated with a BSA (bovine serum albumin)-[¹⁴C]hexanoyl-ceramide complex in the presence of UDP-glucose and then the quantity of [¹⁴C]hexanoyl glucose-ceramides produced is analyzed. The lipids are separated by thin-layer chromatography (TLC) and recovered from the plate by rubbing. The radioactivity is determined by measuring the scintillation linked to incubation of the lipids in scintillant. The background noise is measured by incubating [¹⁴C]hexanoyl-ceramides in a 25 mM KCl/50 mM Tris solution pH 7.4 at 37° C. in the absence of liver extract.

Modulators of the Enzyme:

The present invention also features the use of a modulator of the human enzyme UDP-glucose ceramide glucosyltransferase which can be obtained by one of the above methods, for the preparation of a medicament intended for the preventive and/or curative treatment of acne, or of skin disorders associated with a hyperkeratinization.

A method for the preventive and/or curative treatment of acne, or of skin disorders associated with a hyperkeratinization, is thus described here, the regime or regimen comprising the administration of a therapeutically effective quantity of a modulator of the human enzyme UDP-glucose ceramide glucosyltransferase, to a patient requiring such a treatment.

This invention also features the cosmetic application of a modulator of the human enzyme UDP-glucose ceramide glucosyltransferase for the aesthetic treatment of desquamation problems.

Preferably, the modulator is an inhibitor of the enzyme. The term “inhibitor” refers to a chemical compound or substance which substantially eliminates or reduces the enzymatic activity of UDP-glucose ceramide glucosyltransferase. The term “substantially” means a reduction of at least 25%, preferably of at least 35%, preferably still of at least 50%, and more preferably of at least 70% or 90%. More particularly, it may be a compound which interacts with, and blocks, the catalytic site of the enzyme, such as compounds of the competitive inhibitor type.

A preferred inhibitor interacts with the enzyme in solution at inhibitor concentrations of less than 1 μM, preferably of less than 0.1 μM, preferably still of less than 0.01 μM.

The modulator compound may be an anti-UDP-glucose ceramide glucosyltransferase inhibitory antibody, preferably a monoclonal antibody. Advantageously, such an inhibitory antibody is administered in a quantity sufficient to obtain a plasma concentration of about 0.01 μg per ml to about 100 μg/ml, preferably of about 1 μg per ml to about 5 μg/ml.

The modulator compound may also be a polypeptide, a DNA or RNA anti-sense polynucleotide, an si-RNA or a PNA (“peptide nucleic acid”, polypeptide chain substituted with purine and pyrimidine bases whose spatial structure mimics that of DNA and allows hybridization thereto).

Several UDP-glucose ceramide glucosyltransferase inhibitors are known, and are proposed in particular for the treatment of Gaucher's disease. The invention comprises the administration of such UDP-glucose ceramide glucosyltransferase inhibiting compounds for the preventive and/or curative treatment of acne or skin disorders associated with a hyperkeratinization.

More particularly, without limitation, the following compounds are examples of inhibitors of UDP-glucose ceramide glycosyltransferase:

N-butyldeoxynojirimycin (Miglustat);

D-threo-1-(3′,4′-ethylenedioxy)phenyl-2-palmitoylamino-3-pyrrolidino-1-propanol;

1-threo-1-phenyl-2-decanoylamino-3-morpholino-1-propanol (PDMP);

D-threo-1-phenyl-2-decanoylamino-3-morpholino-1-propanol (D-PDMP);

D-threo-1-phenyl-2-palmitoylamino-3-pyrrolidino-1-propanol (P4);

D-threo-1-phenyl-2-benzyloxycarbonylamino-3-pyrrolidino-1-propanol (PBPP);

D-threo-4′-hydroxy-D-threo-1-phenyl-2-palimitoylamino-3-pyrrolidino-1-propanol (D-threo-4′-hydroxy-P4);

D-threo-1-(3′,4′-methylenedioxy)phenyl-2-palimitoylamino-3-pyrrolidino-1-propanol;

D-threo-1-(3′,4′-ethylenedioxy)phenyl-2-palimitoylamino-3-pyrrolidino-1-propanol;

D-threo-1-(3′,4′-trimethylenedioxy)phenyl-2-palimitoylamino-3-pyrrolidino-1-propanol;

1-threo-1-phenyl-2-hexanoylamino-3-morpholino-1-propanol;

1-threo-1-phenyl-2-heptanoylamino-3-morpholino-1-propanol;

1-threo-1-phenyl-2-octanoylamino-3-morpholino-1-propanol;

1-threo-1-phenyl-2-nonanoylamino-3-morpholino-1-propanol;

1-threo-1-phenyl-2-undecanoylamino-3-morpholino-1-propanol;

1-threo-1-phenyl-2-dodecanoylamino-3-morpholino-1-propanol;

1-threo-1-phenyl-2-tridecanoylamino-3-morpholino-1-propanol;

1-threo-1-phenyl-2-tetradecanoylamino-3-morpholino-1-propanol;

1-threo-1-phenyl-2-pentadecanoylamino-3-morpholino-1-propanol;

1-threo-1-phenyl-2-hexadecanoylamino-3-morpholino-1-propanol;

1-threo-1-phenyl-2-heptadecanoylamino-3-morpholino-1-propanol; and

1-threo-1-phenyl-2-octadecanoylamino-3-morpholino-1-propanol.

Other modulator compounds identified by the screening method described above are also useful.

The modulator compounds are formulated in a pharmaceutical composition, in combination with a pharmaceutically acceptable vehicle. These compositions may be administered for example orally, enterally, parenterally or topically. Preferably, the pharmaceutical composition is applied topically. By the oral route, the pharmaceutical composition may be provided in the form of tablets, gelatin capsules, sugar-coated tablets, syrups, suspensions, solutions, powders, granules, emulsions, suspensions of microspheres or nanospheres or lipid or polymer vesicles allowing controlled release. By the parenteral route, the pharmaceutical composition may be provided in the form of solutions or suspensions for infusion or injection.

By the topical route, the pharmaceutical composition is more particularly useful for the treatment of the skin and the mucous membranes and may be provided in the form of salves, creams, milks, ointments, powders, impregnated pads, solutions, gels, sprays, lotions or suspensions. It may also be provided in the form of suspensions of microspheres or nanospheres or of lipid or polymer vesicles or of polymer patches or hydrogels allowing controlled release. This composition for topical application may be provided in anhydrous form, in aqueous form or in the form of an emulsion. In a preferred embodiment, the pharmaceutical composition is provided in the form of a gel, a cream or a lotion.

The composition may comprise an amount of UGCG modulator ranging from 0.001 to 10% by weight, in particular from 0.01 to 5% by weight relative to the total weight of the composition.

The pharmaceutical composition may additionally contain inert additives or combinations of these additives, such as:

wetting agents;

taste enhancing agents;

preservatives such as para-hydroxybenzoic acid esters;

stabilizing agents;

moisture regulating agents;

pH regulating agents;

osmotic pressure modifying agents;

emulsifying agents;

UV-A and UV-B screening agents;

and antioxidants, such as alpha-tocopherol, butylated hydroxyanisole or butylated hydroxytoluene, Super Oxide Dismutase, Ubiquinol or certain metal chelators.

Legend for the Figures:

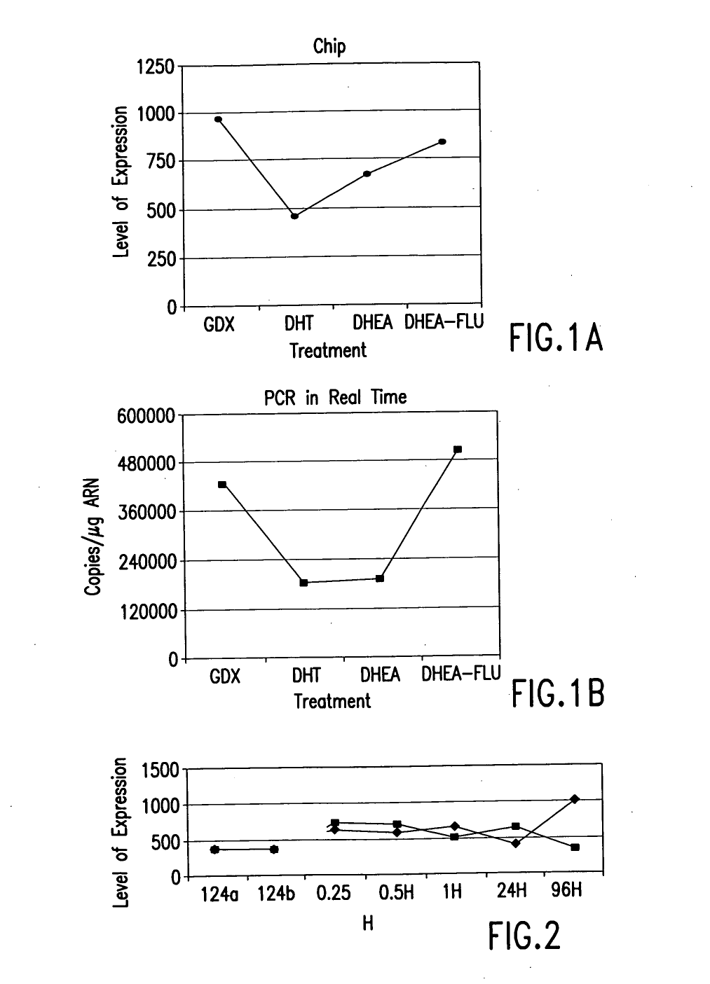

FIGS. 1A and 1B are graphs which show the measurement of the expression of the UGCG gene in gonadectomized male mice treated with the vehicle, DHT, DHEA or the combination of DHEA-Flutamide for a period of 7 days once per day (long-term treatment). The results obtained by the Affymetrix technique (FIG. 1A) were confirmed by the real-time RT-PCR technique (FIG. 1B).

GDX: gonadectomized mice treated with the vehicle.

DHT: gonadectomized mice treated with Dihydrotestosterone (agonist of the androgen receptor).

DHEA: gonadectomized mice treated with Dihydroepiandrosterone (precursor of the steroid hormones; in the preputial glands metabolized to the active androgen).

DHEA-Flu: gonadectomized mice treated with a combination of Dihydroepiandrosterone and Flutamide (antagonist of the androgen receptor; which blocks the effects of the DHT and DHEA agonists).

Level of Expression: Level of Expression of the mRNA:

FIG. 2 is a graph presenting a kinetic study of 15 minutes to 96 hours. In FIG. 2, points 124a and 124b show the level of expression of UGCG of control mice (=non-gonadectomized mice; duplicate) at the 24 hour point. The next points are from gonadectomized mice and indicate the successive times (in hours) of the kinetic study.

Level of Expression: Level of Expression of mRNA:

Square: expression in the gonadectomized mice following treatment with DHT at the time zero.

Diamond: expression in gonadectomized mice without DHT treatment.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Examples

### Experimental Data

### Example 1

### Expression of UDP-Glucose Ceramide Glucosyltransferase (UGCG) in the Human Sebaceous Gland and in the Human Epidermis

Human sebaceous glands were separated from the human epidermis by treatment with dispase and dissection under a binocular lens. Samples of total RNA were prepared from the sebaceous glands and from the epidermis.

The expression of the genes was analyzed on an Affymetrix station (microfluidic model; hybridization oven; scanner; computer) following the protocols provided by the company. Briefly, the total RNA isolated from the tissues is transcribed to cDNA. From the double-stranded cDNA, a cRNA labeled with biotin is synthesized using T7 polymerase and a precursor NTP conjugated to biotin. The cRNAs are then fragmented to small sized fragments. All the molecular biology steps are checked using the Agilent “Lab on a chip” system in order to confirm the good efficiencies of the enzymatic reactions. The Affymetrix chip is hybridized with the biotinylated cRNA, rinsed and then fluorescence labeled using a fluorophore conjugated to streptavidin. After washings, the chip is scanned and the results are calculated using the MAS5 software provided by Affymetrix. An expression value is obtained for each gene as well as the indication of the significance of the value obtained. The calculation of the significance of the expression is based on the analysis of the signals, which are obtained following hybridization of the cRNA of a given gene with an oligonucleotide that is a perfect match compared with an oligonucleotide which contains a single mismatch in the central region of the oligonucleotide (see Table 1).

TABLE 1
measurement of the expression of UDP-glucose ceramide glucosyltransferase in the epidermis
and in the human sebaceous gland through the use of the Affymetrix chip technology.

| | | | | Significance of | Significance of |
|---|---|---|---|---|---|
| | | Expression | Expression | the expression* | the expression* |
| Affymetrix | Name of | in the human | in the human | in the human | in the human |
| identifier | the gene | sebaceous gland | epidermis | sebaceous gland | epidermis |
| 204881_s_at | UDP glucose | 299 | 695 | 1 | 1 |
| | ceramide | | | | |
| | glycosyl- | | | | |
| | transferase | | | | |

*Indicator of the significance of the expression of the gene analyzed in the sample indicated: presence (=1) or absence (=0).

Results:

UGCG is well expressed in both tissues (sebaceous gland, epidermis). Differential analysis from the expression in the human sebaceous gland and the human epidermis shows that the expression is significantly higher in the epidermis (Table 1).

### Example 2

### Expression of UDP-Glucose Ceramide Glucosyltransferase in the Mouse Preputial Gland

A. The mouse preputial glands show differentiation of the sebocyte type and are used as an experimental model for a sebaceous gland. They have a sufficient size to allow isolation of RNA without having recourse to microdissection technologies.

Analysis of the expression of UGCG in the mouse preputial glands was carried out under conditions of deficiencies of steroid hormones (in particular of androgenic hormones) following a gonadectomy. The gonadectomized animals were then treated with physiological quantities of Dihydrotestosterone (DHT) or Dihydroepiandrosterone (DHEA) in order to restore a physiological level of androgenic hormones, or as a control experiment with a DHEA-Flutamide combination in which the Flutamide, an antagonist of the androgen receptors, blocks the effect of DHEA. Comparison of the gene expression under these experimental conditions makes it possible to unambiguously identify the modulation or non-modulation of the gene expression of a gene in question by the androgenic hormones.

The gene expression was analyzed using the Affymetrix technology described above (FIG. 1A) and the results were then confirmed by the real-time PCR technique (FIG. 1B).

The real-time PCR was carried out using the protocols provided by the company Applied Biosystems using the 7900HT Sequence Detection System. The total RNA isolated from the tissues is transcribed (RT) to cDNA and the latter is amplified by PCR (Polymerase Chain Reaction). The progress of the PCR is monitored in real time using fluorescent TaqMan probes which allow precise quantification of the quantity of mRNA of a given gene present in the biological sample at the start.

Result:

The amount of mRNA for UGCG is reduced as a result of a chronic treatment for 7 days with androgens in the preputial gland.

B. Male mice were gonadectomized and treated with the vehicle or DHT. The preputial glands were removed for a period ranging up to 4 days (androgenic treatment alone—observation of a short-term kinetics). The RNA was isolated and the expression of the genes was analyzed by the Affymetrix technique. FIG. 2 represents the relative level of expression of the mRNA as a function of time.

Results:

Gonadectomy (which causes a steroid hormone deficiency) induces a slight induction of the expression of UGCG in the mouse preputial gland.

The mRNA for UGCG in the mouse preputial gland is reduced by a medium-term treatment with DHT (effect visible at 96 hours).

### Example 3

### Formulations

A: Oral Route:

0.2 g tablet

| | |
|---|---|
| D-threo-1-phenyl-2-palmitoylamino-3- | 0.001 g |
| pyrrolidino-1-propanol | |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

B: Topical Route:

(a) Salve

| | | |
|---|---|---|
| 1-Threo-1-phenyl-2-decanoylamino-3- | 0.300 | g |
| morpholino-1-propanol | | |
| Petroleum jelly | qs 100 | g |

(b) Lotion

| | | |
|---|---|---|
| N-butyldeoxynojirimycin | 0.100 | g |
| Polyethylene glycol (PEG 400) | 69.900 | g |
| Ethanol at 95% | 30.000 | g |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An in vitro method for screening candidate compounds for the preventive and/or curative treatment of acne, or skin disorders associated with a hyperkeratinization, comprising determining the capacity of a candidate compound to modulate the expression or activity of UDP-glucose ceramide glucosyltransferase or the expression of its gene or the activity of at least one of its promoters.

2. An in vitro method for screening candidate compounds for the preventive and/or curative treatment of acne or skin disorders associated with a hyperkeratinization as defined by claim 1, comprising the following steps:
a) preparing at least two biological samples or reaction mixtures;
b) bringing one of the samples or reaction mixtures into contact with one or more test compounds;
c) measuring the expression or activity of the protein UDP-glucose ceramide glucosyltransferase, the expression of its gene or the activity of at least one of its promoters, in the biological samples or reaction mixtures;
d) selecting the compounds for which a modulation of the expression or activity of the UDP-glucose ceramide glucosyltransferase, or a modulation of the expression of its gene or a modulation of the activity of at least one of its promoters, is measured in the sample or mixture treated in b), compared with the untreated sample or mixture.

3. The in vitro method as defined by claim 2, wherein the compounds selected in step d) inhibit the expression or the activity of the protein UDP-glucose ceramide glucosyltransferase, the expression of its gene or the activity of at least one of its promoters.

4. The in vitro method as defined by claim 2, wherein the biological samples are cells transfected with a reporter gene that is operably linked to all or part of the promoter of the gene encoding the protein UDP-glucose ceramide glucosyltransferase, and in that step c) comprises measuring the expression of the said reporter gene.

5. The in vitro method as defined by claim 2, wherein the biological samples are cells expressing the gene encoding the protein UDP-glucose ceramide glucosyltransferase, and in that step c) comprises measuring the expression of the said gene.

6. The in vitro method as defined by claim 4, in which the cells are keratinocytes or sebocytes.

7. The in vitro method as defined by claim 5, in which the cells are cells transformed with a heterologous nucleic acid encoding UDP-glucose ceramide glucosyltransferase.

8. The in vitro method as defined by claim 2, in which the expression of the gene is determined by measuring the level of transcription of the said gene.

9. The in vitro method as defined by claim 2, in which the expression of the gene is determined by measuring the level of translation of the said gene.

10. The in vitro method as defined by claim 2, wherein step a) comprises preparing reaction mixtures each comprising an enzyme UDP-glucose ceramide glucosyltransferase and a substrate of the enzyme, and in that step c) comprises measuring the enzyme activity.
